(19) United States
(12) Patent Application Publication
(10) Pub. No.: US 2014/0371726 A1
(43) Pub. Date: Dec. 18, 2014

(54) INTELLIGENT FAT SUCTION CANNULA

(71) Applicant: Kwon Lee, II, Gyeonggi-do (KR)

(72) Inventor: Kwon Lee, II, Gyeonggi-do (KR)

(73) Assignee: Chungwoo Medical Co., Ltd., Seoul (KR)

(21) Appl. No.: 14/381,087

(22) PCT Filed: Feb. 29, 2012
(86) PCT No.: PCT/KR2012/001524

§ 371 Date: Aug. 26, 2014

(51) Int. Cl. A61M 1/00; A61B 17/32

(52) U.S. Cl. CPC A61M 1/0025; A61M 1/0041; A61B 17/320068; A61M 2205/3368; A61M 2202/08; A61M 2205/3327; A61B 2017/320072
USPC 604/542; 604/319

(57) ABSTRACT

Disclosed herein is an intelligent cannula apparatus for suctioning fat. The intelligent cannula apparatus includes a cannula, a temperature sensor and a handpiece. The cannula has a hollow tubular shape. A plurality of fat suction holes are formed in a front end of the cannula. The cannula generates, from an outer circumferential surface thereof, a high frequency for dissolving fat and facilitating suction of the fat and measures impedance from the high frequency, thus determining a target body part. The temperature sensor is installed in the cannula to measure a temperature of the body part in accordance with the generation of the high frequency. The handpiece is coupled to the cannula.

### TECHNICAL FIELD

The present invention relates, in general, to cannulas which are inserted into fat regions of human bodies and used for suctioning fat and, more particularly, to an intelligent cannula apparatus for suctioning fat which is configured such that, after the temperature and motion of a cannula inserted into a human body are sensed and a target body part is determined by measuring impedance using a high-frequency current flowing through the human body, the output rate of the high frequency is automatically controlled based on the sensed and determined information, whereby constant surgical procedure effects can be provided regardless of an operator, and damage of tissues, such as blood vessels, a muscular coat, a skin layer, etc., of the body part can be minimized.

### BACKGROUND ART

Recently, overall improvements in standards of living have lead to an increase in the intake of meat, and the development of transportation has reduced the amount of time people spend exercising by walking, thus the accumulation rate of animal fat, which is not easily dissolved in the human body, has increased. As a result, the percentage of the population that is obese is increasing. Obesity is a kind of disease and is a cause of various adult illnesses such as arteriosclerosis, diabetes, etc.

Along with this change, medical advances have increased the use of liposuction, which is a process to artificially remove fat excessively accumulated in the body and thus changes the form of the body into a slim body type for the purposes of curing obesity and enhancing beauty.

Such liposuction is typically performed in such a way that, inserted into a fat layer of the human body, a cannula having a tubular shape suctions fat cells and removes them while moving forwards and backwards.

However, in a fat suction apparatus using the conventional cannula, to suction fat from the fat layer in the skin of a patient, an operator must intensively operate on a region where a large amount of fat is accumulated, while moving the cannula forwards and backwards after it is inserted into the fat layer of the patient's body. Hence, depending on the skill level and experience of the operator, the effects of the surgical procedure vary significantly.

Moreover, the structure of blood vessels, muscular coats, skin layers, etc. in the human body varies according to body regions. However, the conventional fat suction apparatus suctions fat at a constant speed and in the same operation manner regardless of a target region of the patient's body. Therefore, if an unskilled operator performs a surgical procedure using the conventional art, the blood vessels, the muscular coat, the skin layer or the like in the target region of the patient's body may be easily damaged. As a result, side effects may be caused after the surgical procedure has been conducted.

### DISCLOSURE

### Technical Problem

Accordingly, the present invention has been made keeping in mind the above problems occurring in the prior art, and an object of the present invention is to provide an intelligent cannula apparatus for suctioning fat which is configured such that an output rate of high frequency can be automatically adjusted depending on the target body part, whereby constant surgical procedure effects can be provided regardless of an operator.

Another object of the present invention is to provide an intelligent cannula apparatus for suctioning fat which can minimize damage of tissues, such as blood vessels, a muscular coat, a skin layer, etc., of the body part, thus preventing side effects after the surgical procedure has been completed.

### Technical Solution

In order to accomplish the above objects, the present invention provides an intelligent cannula apparatus for suctioning fat, including: a cannula having a hollow tubular shape, with a plurality of fat suction holes formed in a front end of the cannula, the cannula generating, from an outer circumferential surface thereof, a high frequency for dissolving fat and facilitating suction of the fat, the cannula measuring impedance from the high frequency and thus determining a target body part; a temperature sensor installed in the cannula, the temperature sensor measuring a temperature of the body part in accordance with the generation of the high frequency; and a handpiece coupled to the cannula.

The intelligent cannula apparatus may further include a motion sensor installed in the handpiece, the motion sensor sensing motion of the cannula.

The front end of the cannula in which the fat suction holes are formed may have a streamlined shape.

Furthermore, a portion of the cannula in which the fat suction holes are not formed may be coated with an insulation layer.

The intelligent cannula apparatus may further include a control unit connected to: a temperature sensing unit for sensing the temperature of the cannula using the temperature sensor; a motion sensing unit for sensing the motion of the cannula using the motion sensor; and an impedance measurement unit for measuring impedance using a high-frequency current flowing from the cannula through a human body and determining the target body part.

The control unit may determine the body part in consideration of the sensed temperature and motion of the cannula and the measured impedance and automatically control a high-frequency output rate in accordance with the sensed temperature and motion and the measure impedance.

The control unit may be disposed in the handpiece. Alternatively, the control unit may be disposed in a housing of a fat suction apparatus.

The control unit may further include: a temperature signal amplifier amplifying a temperature signal sensed by the temperature sensor; a motion signal amplifier amplifying a motion signal sensed by the motion sensor; and an impedance signal amplifier amplifying an impedance signal.

The handpiece may be connected to the housing of the fat suction apparatus, with a fat suction pump installed in the housing. The housing may be provided both with a fat storage tank for use in collecting fat suctioned through the fat suction tube and with a manipulator for use in turning on or off the cannula apparatus or manually adjusting intensity of suction force of the fat suction pump or the high-frequency output rate.

A display unit may be provided on the housing. The display unit may display current operation conditions of the cannula apparatus, the temperature of the cannula, the impedance measured by the impedance measurement unit, the body part determined by the impedance measurement unit, and a current high-frequency output rate.

### Advantageous Effects

An intelligent cannula apparatus for suctioning fat according to the present invention is configured such that, after the temperature and motion of a cannula inserted into a human body are sensed and a body part to be operated is determined by measuring impedance using high-frequency current flowing through the human body, the output rate of high frequency is automatically controlled based on the sensed and determined information. Thereby, constant surgical procedure effects can be provided regardless of an operator, and damage of tissues, such as blood vessels, a muscular coat, a skin layer, etc., of the body part can be minimized.

### DESCRIPTION OF DRAWINGS

FIG. 1 is a perspective view illustrating an intelligent cannula apparatus for suctioning fat according to an embodiment of the present invention;

FIG. 2 is an exploded perspective view illustrating the cannula apparatus and a temperature sensor according to the embodiment of the present invention;

FIG. 3 is a block diagram showing an operation control structure of the cannula apparatus according to the embodiment of the present invention; and

FIG. 4 is a view showing the construction of a fat suction machine to which the cannula apparatus according to the present invention is connected.

### BEST MODE

Hereinafter, a preferred embodiment of the present invention will be described in detail with reference to the attached drawings. Those skilled in the art will appreciate that various modifications are possible, and the present invention is not limited to the following embodiment.

The embodiment of the present invention is provided to fully describe the present invention to those having ordinary knowledge in the art to which the present invention pertains. Accordingly, in the drawings, the shapes and sizes of elements may be exaggerated for the sake of clearer description. Reference should now be made to the drawings, in which the same reference numerals are used throughout the different drawings to designate the same or similar components.

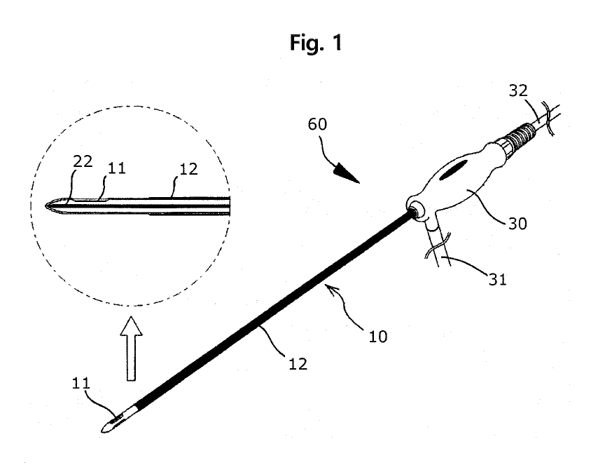

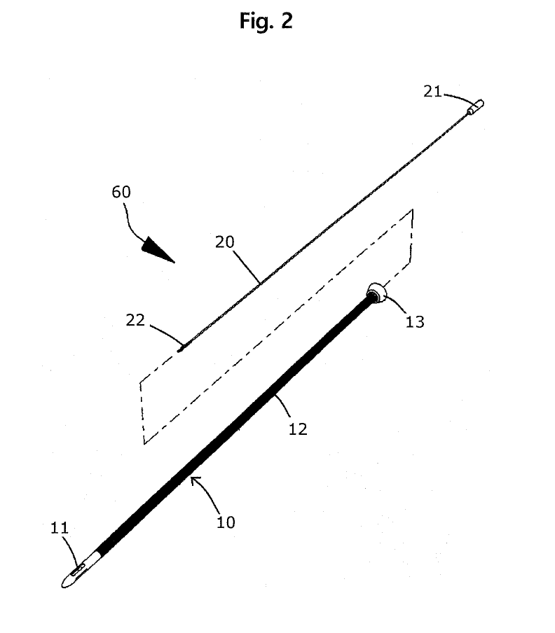

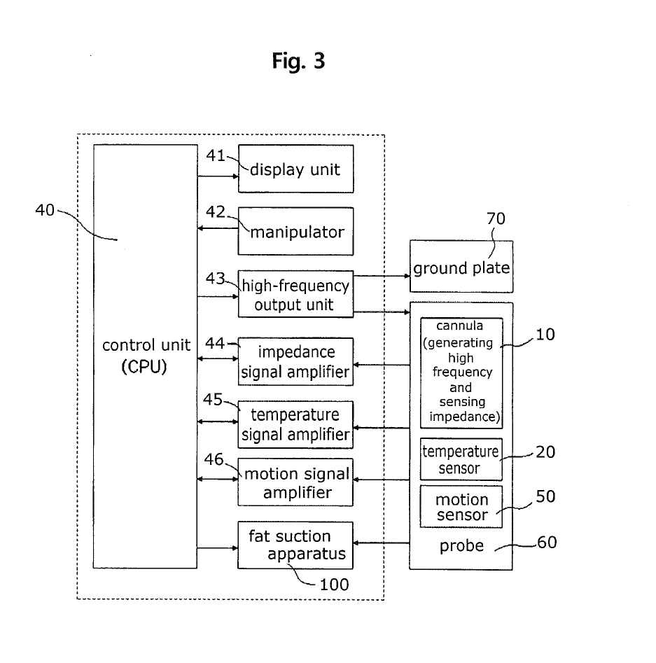

As shown in FIGS. 1 through 4, a cannula apparatus for suctioning fat according to an embodiment of the present invention includes a cannula 10, a rod-shaped temperature sensor 20 and a handpiece 30, thus forming a probe 60. The cannula 10 has a hollow tubular body. A plurality of fat suction holes 11 are formed in a front end of the cannula 10. The cannula 10 generates a high frequency from an outer circumferential surface of the tubular body so as to dissolve fat and thus facilitate suction of the fat. The cannula 10 includes an impedance measurement unit (not shown) which transmits the high frequency to the human body and measures impedance, thus determining a target body part. The temperature sensor 20 is disposed in the cannula 10 so as to measure the temperature of the body part in accordance with the generation of high frequency. The handpiece 30 is coupled to the cannula 10.

In detail, to make it easy to penetrate the subcutaneous tissue of a patient with the cannula 10, a first end of the cannula 10 has a streamlined shape in such a way that the diameter thereof is gradually reduced from a proximal side to a distal side. A coupling part 13 for use in connection to the handpiece 30 is provided on a second end of the cannula 10. Made of insulation material, an insulation layer 12 is formed around a portion of the cannula 10 in which the fat suction holes 11 are not formed.

The insulation layer 12 may be formed by directly applying insulation material to the outer circumferential surface of the cannula 10 or, alternatively, by manufacturing a separate insulation sheet using insulation material and then attaching the insulation sheet to the outer circumferential surface of the cannula 10.

A connector 21 for use in electrical connection is provided on a rear end of the temperature sensor 20. The temperature sensor 20 measures the temperature of the target body part in accordance with generation of high frequency. The output rate of high frequency of the cannula 10 can be controlled depending on the measure temperature so as to prevent the tissues of the human body from being burned by heat which is generated by an excessively high output rate of high frequency. Furthermore, by virtue of the temperature sensor 20, the temperature of the cannula 10 can be maintained at a constant temperature at which dissolution of fat in the target body part can be optimized.

The handpiece 30 preferably has a streamlined curved shape to enable an operator to easily grip it when conducting a surgical procedure. The handpiece 30 is connected to the coupling part 13 of the cannula 10 and includes a fat suction tube 31 through which fat suctioned by the cannula 10 is discharged out of the cannula apparatus, and a cable 32 which is electrically connected to the cannula 10 and the connector 21 of the temperature sensor 20.

When electric current is applied to the cannula 10 through the cable 32, a high frequency is generated from the outer circumferential surface of the cannula 10. Simultaneously, the impedance measurement unit senses impedance from the high frequency, and the temperature sensor 20 installed in the cannula 10 senses the temperature of the cannula 10.

Furthermore, a motion sensor 50 for use in measuring acceleration of an object or the intensity of an impact is installed in the handpiece 30. The motion sensor 50 senses conditions of the surgical procedure, for example, speed or direction of forward or backward movement of the cannula 10, determines the target body part, and controls the high frequency output depending on the determined result.

A control unit 40 is installed in the handpiece 30. The control unit 40 is connected to a temperature sensing unit (not shown) which senses the temperature of the cannula 10 using the temperature sensor 20, a motion sensing unit (not shown) which senses the motion of the cannula 10 using the motion sensor 50, and the impedance measurement unit which measures the impedance using current flowing from the cannula 10 through the human body and determines the target body part.

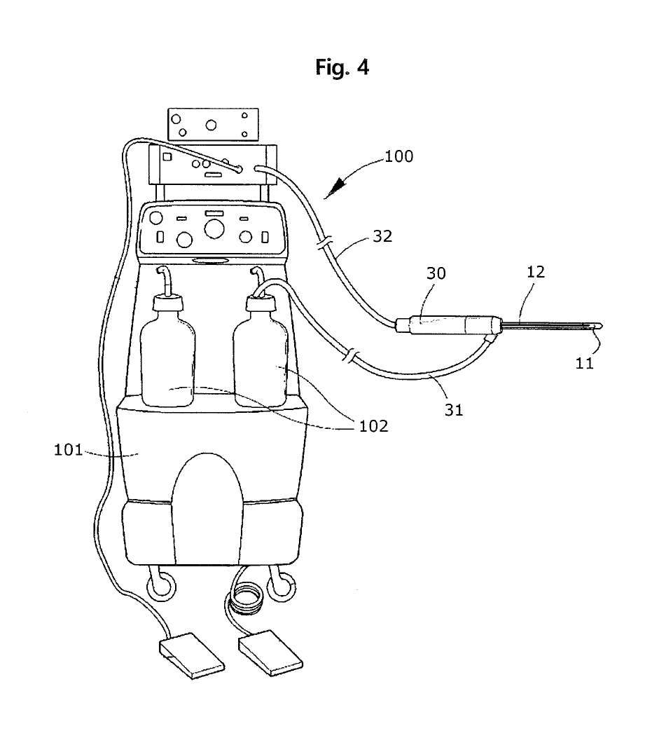

The control unit 40 may be installed in a housing 101 of a fat suction apparatus shown in FIG. 4. Given the impedance and the temperature and the motion of the cannula 10 that are measured or sensed by the above-mentioned method, the control unit 40 determines the target body part and, depending on the determined result, then controls the high frequency output rate using a high-frequency output unit 43.

Furthermore, the control unit 40 includes a temperature signal amplifier 45 which amplifies a temperature signal sensed by the temperature sensor 20, a motion signal amplifier 46 which amplifies a motion signal sensed by the motion sensor 50, and an impedance signal amplifier 44 which amplifies an impedance signal. By virtue of such construction, the control unit 40 can effectively determine the target body part despite small changes in conditions.

The handpiece 30 is connected to the housing 101 of the fat suction apparatus 100 in which a fat suction pump (not shown) is installed. The housing 101 has therein a fat storage tank 102 in which fat suctioned by the fat suction tube 31 is stored, and a manipulator 42 which is used in turning on or off the cannula apparatus or manually adjusting the intensity of suction force of the fat suction pump, the high-frequency output rate of the high-frequency output unit 43, etc., as needed.

Furthermore, a display unit 41 may be provided on the housing 101 so as to display current operation conditions of the cannula apparatus, the temperature of the cannula 10, the impedance measured by the impedance measurement unit, the target body part determined by the impedance measurement unit, a current high-frequency output rate, etc.

Reference numeral 70 denotes a ground contact part for use in preventing malfunction caused by an electrical accident resulting from overcurrent or the like. The ground contact part is electrically connected to the fat suction apparatus 100 and the control unit 40.

### MODE FOR INVENTION

Hereinafter, the operation of the cannula apparatus for suctioning fat according to an embodiment of the present invent will be described in detail.

When the cannula apparatus is turned on by means of the manipulator 46, the fat suction pump is operated so that vacuum suction force is generated through in the fat suction tube 31. In addition, a high frequency is generated from the outer circumferential surface of the cannula 10.

Subsequently, the operator grips the cannula 10 and inserts it into a target body part. Thereafter, the operator repeatedly moves the cannula 10 forwards and backwards. Then, fat is suctioned from the target body part through the suction hole 11 and collected in the fat storage tank 102.

During this process, the temperature sensor 20 senses the temperature of the cannula 10, the motion sensor 50 senses the motion of the cannula 10, and the impedance measurement unit measures impedance.

The impedance measurement unit measures impedance from a high-frequency current generated from the cannula 10. The impedance may be measured by measuring a flow rate of the high frequency. In another embodiment, the impedance may be measured by measuring a value of high frequency when the high frequency is suddenly interrupted.

Subsequently, given all of the sensed temperature and motion of the cannula 10 and the measured impedance, the control unit 40 determines a target body part. The control unit 40 automatically controls the high-frequency output rate of the high-frequency output unit 43 in accordance with the corresponding body part such that the optimum fat suction force is provided, whereby damage of tissues of the body part can be minimized.

### INDUSTRIAL APPLICABILITY

Although the preferred embodiment of the present invention has been disclosed, those skilled in the art will appreciate that various modifications, additions and substitutions are possible, without departing from the scope and spirit of the invention.

Therefore, the embodiment disclosed in this specification is only for illustrative purposes rather than limiting the technical spirit of the present invention.

The scope of the present invention must be defined by the accompanying claims, and all technical spirits that are in the equivalent range to the claims must be regarded as falling within the scope of the present invention.

1. An intelligent cannula apparatus for suctioning fat, comprising:
a cannula having a hollow tubular shape, with a plurality of fat suction holes formed in a front end of the cannula, the cannula generating, from an outer circumferential surface thereof, a high frequency for dissolving fat and facilitating suction of the fat, the cannula measuring impedance from the high frequency and thus determining a target body part;
a temperature sensor installed in the cannula, the temperature sensor measuring a temperature of the body part in accordance with the generation of the high frequency; and
a handpiece coupled to the cannula.

2. The intelligent cannula apparatus of claim 1, further comprising
a motion sensor installed in the handpiece, the motion sensor sensing motion of the cannula.

3. The intelligent cannula apparatus of claim 1, wherein the front end of the cannula in which the fat suction holes are formed has a streamlined shape.

4. The intelligent cannula apparatus of claim 1, wherein a portion of the cannula in which the fat suction holes are not formed is coated with an insulation layer.

5. The intelligent cannula apparatus of claim 2, further comprising
a control unit connected to: a temperature sensing unit for sensing the temperature of the cannula using the temperature sensor; a motion sensing unit for sensing the motion of the cannula using the motion sensor; and an impedance measurement unit for measuring impedance using a high-frequency current flowing from the cannula through a human body and determining the target body part.

6. The intelligent cannula apparatus of claim 5, wherein the control unit determines the body part in consideration of the sensed temperature and motion of the cannula and the measured impedance and automatically controls a high-frequency output rate in accordance with the sensed temperature and motion and the measure impedance.

7. The intelligent cannula apparatus of claim 5, wherein the control unit is disposed in the handpiece.

8. The intelligent cannula apparatus of claim 5, wherein the control unit is disposed in a housing of a fat suction apparatus.

9. The intelligent cannula apparatus of claim 6, wherein the control unit further comprises:
a temperature signal amplifier amplifying a temperature signal sensed by the temperature sensor;
a motion signal amplifier amplifying a motion signal sensed by the motion sensor; and
an impedance signal amplifier amplifying an impedance signal.

10. The intelligent cannula apparatus of claim 8, wherein the handpiece is connected to the housing of the fat suction apparatus, with a fat suction pump installed in the housing, and
the housing is provided both with a fat storage tank for use in collecting fat suctioned through the fat suction tube and with a manipulator for use in turning on or off the cannula apparatus or manually adjusting intensity of suction force of the fat suction pump or the high-frequency output rate.

11. The intelligent cannula apparatus of claim 10, wherein a display unit is provided on the housing, the display unit displaying current operation conditions of the cannula apparatus, the temperature of the cannula, the impedance measured by the impedance measurement unit, the body part determined by the impedance measurement unit, and a current high-frequency output rate.
